(19) United States
(12) Patent Application Publication
(10) Pub. No.: US 2007/0203119 A1
(43) Pub. Date: Aug. 30, 2007

(54) Combination Therapy For The Treatment Of Schizophrenia

(76) Inventors: Philippe Danjou, London (GB); Peter Haynes Hutson, Saffron Walden (GB)

Correspondence Address: MERCK AND CO., INC, P O BOX 2000, RAHWAY, NJ, 07065-0907, US

(21) Appl. No.: 11/659,672

(22) PCT Filed: Aug. 1, 2005
(86) PCT No.: PCT/GB05/50123

§ 371 Date: Feb. 7, 2007

(30) Foreign Application Priority Data
Aug. 9, 2004 (GT) 0417702.8

(51) Int. Cl. A61K 31/55; A61K 31/519; A61K 31/47; A61K 31/496; A61K 31/551

(52) U.S. Cl. 514/217.05; 514/253.06; 514/220; 514/259.41; 514/217.07; 514/314

(57) ABSTRACT

The present invention relates to a combination of a NK3 receptor antagonist with a 5HT2A receptor antagonist and/or a D2 receptor antagonist, and the use of the combination in treating schizophrenia and associated conditions.

The present invention relates to a combination of a NK3 receptor antagonist with a 5HT2A receptor antagonist and/or a D2 receptor antagonist, and the use of the combination in treating schizophrenia and associated conditions.

Schizophrenia is a psychopathic disorder of unknown origin, which usually appears for the first time in early adulthood and is marked by a number of characteristics, psychotic symptoms, progression, phasic development and deterioration in social behaviour and professional capability in the region below the highest level ever attained. Characteristic psychotic symptoms are disorders of thought content (multiple, fragmentary, incoherent, implausible or simply delusional contents or ideas of persecution) and of mentality (loss of association, flight of imagination, incoherence up to incomprehensibility), as well as disorders of perceptibility (hallucinations), of emotions (superficial or inadequate emotions), of self-perception, of intentions and impulses, of interhuman relationships, and finally psychomotoric disorders (such as catatonia). Other symptoms are also associated with this disorder. (See, American Statistical and Diagnostic Handbook).

The mammalian peptide Neurokinin B (NKB) belongs to the Tachykinin (TK) peptide family which also include Substance P (SP) and Neurokinin A (NKA). Pharmacological and molecular biological evidence has shown the existence of three subtypes of TK receptor (NK1, NK2 and NK3) and NKB binds preferentially to the NK3 receptor although it also recognizes the other two receptors with lower affinity (Maggi et al., 1993, J. Auton. Pharmacol., 13, 23-93).

Selective peptidic NK3 receptor antagonists are known (Drapeau, 1990 Regul. Pept., 31, 125-135), and findings with peptidic NK3 receptor agonists suggest that NKB, by activating the NK3 receptor, has a key role in the modulation of neural input in airways, skin, spinal cord and nigro-striatal pathways (Myers and Undem, 1993, J. Phisiol., 470, 665-679; Counture et al., 1993, Regul. Peptides, 46, 426-429; Mccarson and Krause, 1994, J. Neurosci., 14 (2), 712-720; Arenas et al. 1991, J. Neurosci., 11, 2332-8).

Non-peptide antagonists for the tachykinin NK3 receptor are also known, see, for instance, published US patent application no. U.S. Pat. No. 5,741,910 (Sanofi) and published International patent application WO 97/19926 (SmithKline Beecham S.p.a.).

Clinical studies have indicated the effectiveness of NK3 receptor antagonists in the treatment of schizophrenia and associated conditions. However, due to the highly complex nature of schizophrenia and related disorders, it is likely that a significant patient population will not respond to treatment with a NK3 antagonist alone.

Schizophrenia may conventionally be treated with drugs known as neuroleptics. In many cases, the symptoms of schizophrenia can be treated successfully with so-called “classical” neuroleptic agents such as haloperidol. Classical neuroleptics are generally antagonists at dopamine D2 receptors, see, for example, Davis and Chen, J. Clin. Psychopharmacol. 2004; 24: 192-208, and Farde et al., Arch. Gen. Psychiatry (1988) 45:71-76.

However, certain patients may display undesired extrapyramidal and other side effects and there thus remains the need to find alternative therapies for the treatment of schizophrenia.

Notwithstanding their beneficial antipsychotic effects, classical neuroleptic agents, such as haloperidol, are frequently responsible for eliciting acute extrapyramidal symptoms (movement disorders) and neuroendocrine (hormonal) disturbances. These side effects, which plainly detract from the clinical desirability of classical neuroleptics, are believed to be attributable to D2 receptor blockade in the striatal region of the brain.

5HT2A receptor antagonists may also be used in the treatment of schizophrenia, see, for example, Mortimer and Barnes, “Serotonergic Mechanisms in Antipsychotic Treatment”, Marcel Dekker, Inc., New York (1996): 311-330.

Some drugs used in the treatment of schizophrenia may display dual 5HT2A and D2 receptor antagonism. Examples include risperidone, ziprasidone, iloperidone, melperone and olanzapine.

Combination therapy for schizophrenia is known. For example, published International patent application WO 2004/058722 (Arena Pharmaceuticals, Inc.) discloses a method for treating, inter alia, schizophrenia by administering a dopamine D2 receptor antagonist and a 5HT2A inverse agonist disclosed therein.

Published International patent application WO 2004/039322 (Mi-icro, Inc.) discloses a method of treating schizophrenia with a combination comprising a drug that blocks 5HT2A receptors, a drug that blocks D2 receptors and a drug that blocks dopamine reuptake.

While the benefits of combinative therapies are well known, such therapies have either not been fully exploited or have not been successfully applied to date in the treatment of schizophrenia. Thus, there is a need in the art for more effective therapies for the treatment of schizophrenia. The use of multiple compounds to treat an indication can increase the beneficial effects while reducing the presence of side effects, especially when the amount of each component is lower when used in combination than when used alone. It is possible that each component may be used in doses that would be sub-therapeutic if used alone.

It has now surprisingly been found that the use of a NK3 receptor antagonist in combination with a 5HT2A receptor antagonist and/or a D2 receptor antagonist is effective in the treatment of schizophrenia, associated conditions, and the symptoms thereof.

Thus, the present invention provides, in a first aspect, a combination comprising a NK3 receptor antagonist, or a pharmaceutically acceptable derivative thereof, a 5HT2A receptor antagonist, or a pharmaceutically acceptable derivative thereof, and/or a D2 receptor antagonist, or a pharmaceutically acceptable derivative thereof.

When referred to herein, the term “a NK3 receptor antagonist, a 5HT2A receptor antagonist and/or a D2 receptor antagonist” is understood to mean a NK3 receptor antagonist and a 5HT2A receptor antagonist, a NK3 receptor antagonist and a D2 receptor antagonist, and a NK3 receptor antagonist and a 5HT2A receptor antagonist and a D2 receptor antagonist. Thus, for example, in the first aspect of the present invention, there is provided the combination comprising a NK3 receptor antagonist, or a pharmaceutically acceptable derivative thereof, and a 5HT2A receptor antagonist, or a pharmaceutically acceptable derivative thereof, the combination comprising a NK3 receptor antagonist, or a pharmaceutically acceptable derivative thereof, and a D2 receptor antagonist, or a pharmaceutically acceptable derivative thereof, and the combination comprising a NK3 receptor antagonist, or a pharmaceutically acceptable derivative thereof, and a 5HT2A receptor antagonist, or a pharmaceutically acceptable derivative thereof, and a D2 receptor antagonist, or a pharmaceutically acceptable derivative thereof.

By pharmaceutically acceptable derivative is meant any pharmaceutically acceptable salt, solvate, ester or amide, or salt or solvate of such ester or amide, of the NK3 receptor antagonist, the 5HT2A receptor antagonist and/or the D2 receptor antagonist, or any other compound which upon administration to the recipient is capable of providing (directly or indirectly) the NK3 receptor antagonist, the 5HT2A receptor antagonist or the D2 receptor antagonist or an active metabolite or residue thereof.

The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or prodrug formulations. Depending on the particular functionality of the compound of the present invention, pharmaceutically acceptable salts of these compounds include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide. These salts may be prepared by standard procedures, e.g. by reacting a free acid with a suitable organic or inorganic base. Where a basic group is present, such as amino, an acidic salt, i.e. hydrochloride, hydrobromide, acetate, palmoate, and the like, can be used as the dosage form.

Also, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

NK3 receptor antagonists may be identified using the tests disclosed in published US patent application no. US2004/002504 (Merck Sharp & Dohme Limited).

Suitable NK3 receptor antagonists for use according to the invention include, for example, those disclosed in published US patent application no. U.S. Pat. No. 5,741,910 (Sanofi) and published International patent application WO 97/19926 (SmithKline Beecham S.p.a.).

Other suitable NK3 receptor antagonists include those disclosed in published International patent applications WO 2004/050626 and WO 2004/050627 (both SmithKline Beecham Corporation) and published US patent application no. US 2004/0002504 (Merck Sharp & Dohme Limited).

Further suitable NK3 receptor antagonists include those disclosed in published International patent application WO 2004/072045 (Merck Sharp & Dohme Limited).

Preferred NK3 receptor antagonists are talnetant, osanetant, 3-(4-(1-methylethyl)piperazin-1-ylmethyl)-2-phenyl-quinoline-4-carboxylic acid azepan-1-ylamide, 3-(4-methylethyl)piperazin-1-ylmethyl)-2-phenyl-quinoline-4-carboxylic acid N′-(1-phenyl-propyl)-hydrazide, methyl 2-{[3-methyloxy-2-phenyl-4-quinolinyl]carbonyl}-1-phenylhydrazinecarboxylate, N′-ethyl-3-methyloxy-N′,2-diphenyl-4-quinolinecarbohydrazide, 3-methyloxy-2-phenyl-N′-(1-phenylpropyl)-4-quinolinecarbohydrazide, 3-methyl-N′,N′,2-triphenyl-4-quinolinecarbohydrazide, N′,3-dimethyl-N′,2-diphenyl-4-quinolinecarbohydrazide, 3-methyl-N′,2-diphenyl-N′-phenylmethyl-4-quinolinecarbohydrazide, N-hexahydrocyclopenta[c]pyrrol-2(1H)-yl-3-methyl-2-phenyl-4-quinolinecarboxamide, N′-cyclopentyl-3-methyl-2-phenyl-N′-propyl-4-quinolinecarbohydrazide, N′-cyclohexyl-3-{[4-(1-methylethyl)-1-piperazinyl]methyl}-2-phenyl-4-quinolinecarbohydrazide, N-hexahydro-1H-azepin-1-yl-3-methyl-2-phenyl-4-quinolinecarboxamide, and their pharmaceutically acceptable salts.

The NK3 receptor antagonists may be used alone or in combination with each other.

5HT2A receptor antagonists may be identified using the methodology described in Fletcher et al., J. Med. Chem., 2002, 45, 492-503.

Suitable 5HT2A receptor antagonists for use according to the invention include, for example, olanzapine, risperidone, ziprasidone, pizotifen, iloperidone, ritanserin, ketanserin, cyproheptadine, aripiprazole, arylipiperazines, risperidone metabs (Sepracor), ACP-103 (ACADIA), AT-1015 (Ajinomoto), Org-5222 (Akzo Nobel), AR-116081 (Arena), MDL-100907 (Aventis), Pharmaprojects No. 2865 (Aventis), RP 71602 (Aventis), IT-657 (Bristol-Myers Squibb), F-94116-CN (Faes), GMC-283 (Merck KGaA), EMR-62218 (Merck KGaA) and Pharmaprojects No. 5350 (Servier), and pharmaceutically acceptable salts thereof.

Other suitable 5HT2A receptor antagonists are disclosed in published International patent application nos. WO 99/11619, WO 99/11641, WO 99/47511, WO 00/04017, WO 00/05229, WO 00/43362 and WO 01/74794 (all Merck Sharp & Dohme Limited).

Further examples of suitable 5HT2A antagonists include those disclosed in published International Application WO 2004/101518 (Merck Sharp & Dohme Limited).

Further examples of suitable 5HT2A antagonists include the compounds of formula (VII): 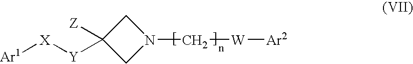
wherein n is 0, 1, 2 or 3;

W is CH₂, CO, CHF or CH(OH);

X is SO₂, CO or CH(OH);

Y is CH₂, CHF or CF₂;

Z is H, F or OH;

Ar¹ is phenyl optionally bearing up to 3 substituents selected from halogen, CN, NO₂, R¹, OR², COR², CO₂R², OCOR¹, SR², S(O)ₜR¹ where t is 1 or 2, N(R²)₂, CON(R²)₂, NR²COR¹ and SO₂N(R²)₂;

Ar² is phenyl or heteroaryl, said heteroaryl having 5 or 6 ring atoms of which one, two or three are selected from N, O and S but not more than one of which is O or S, said heteroaryl optionally being benzo-fused, and said phenyl or heteroaryl optionally bearing up to 3 substituents selected from halogen, CN, NO₂, R¹ and OR²;

R¹ is a hydrocarbon group comprising up to 6 carbon atoms optionally bearing up to 5 fluorine substituents; and

R² is R¹ or H; or two R² groups attached to the same nitrogen atom may complete a morpholine or thiomorpholine ring, or a 5- or 6-membered heterocycle wherein the remaining ring atoms are selected from C and N to a maximum of 4 ring nitrogens in total;

and the pharmaceutically acceptable salts thereof.

Specific examples of compounds in accordance with formula (VII) include:
1-[2-(2,4-difluorophenyl)ethyl]-3-{[(4-fluorophenyl)sulfonyl]methyl}azetidine;
1-(4-fluorophenyl)-2-(3-{[(4-fluorophenyl)sulfonyl]methyl}azetidin-1-yl)ethanone;
1-(4-fluorophenyl)-4-(3-{[(4-fluorophenyl)sulfonyl]methyl}azetidin-1-yl)butan-1-one hydrochloride;
2-(3-{[(2,4-difluorophenyl)sulfonyl]methyl}azetidin-1-yl)-1-(4-fluorophenyl)ethanone hydrochloride;
2-(3-fluoro-3-{[(4-fluorophenyl)sulfonyl]methyl}azetidin-1-yl)-1-(4-fluorophenyl)ethanone hydrochloride;
1-[2-(2,4-difluorophenyl)ethyl]-3-{fluoro[(4-fluorophenyl)sulfonyl]methyl}azetidine hydrochloride;
3-{difluoro[(4-fluorophenyl)sulfonyl]methyl}-1-[2-(2,4-difluorophenyl)ethyl]azetidine hydrochloride;
6-fluoro-3-[(3-{[(4-fluorophenyl)sulfonyl]methyl}azetidin-1-yl)methyl]-1,2-benzisothiazole hydrochloride;
6-fluoro-3-[(3-{[(4-fluorophenyl)sulfonyl]methyl}azetidin-1-yl)methyl]-1,2-benzisoxazole hydrochloride; and
1-(4-fluoro-2-methylphenyl)-2-(3-{[(4-fluorophenyl)sulfonyl]methyl}azetidin-1-yl)ethanone.

The compounds of formula (VII) may be obtained by reaction of compounds of formula (1a) with compounds of formula (2): 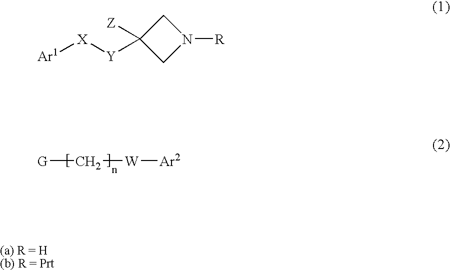
wherein G is a leaving group such as mesylate, tosylate or halide (especially bromide) and n, W, X, Y, Z, Ar¹ and Ar² have the same meanings as before. The reaction takes place under typical alkylation conditions, e.g. in a solvent such as DMF, acetonitrile, acetone, or 2-butanone at ambient or moderately elevated temperatures (e.g. about 70-90° C.), in the presence of a base such as potassium carbonate.

Alternatively, compounds of formula (VII) in which n is 1 or more may be formed by reacting amines (1a) with aldehydes (3):
Ar²—W—(CH₂)ₘ—CHO (3)
where m is 0, 1 or 2 and W and Ar² have the same meanings as before. The reaction takes place in the presence of sodium cyanoborohydride in alcoholic solution (eg methanol) at ambient temperature.

The amines (1a) may be preformed or formed in situ from compounds (1b) where Prt is a protecting group. Preferred protecting groups include diphenylmethyl (which may be removed by hydrogenation over a Pd(OH)₂ catalyst) and t-butoxycarbonyl (BOC) (which may be removed by treatment with acid, e.g. trifluoroacetic acid or HCl in aqueous dioxan).

Compounds (1b) in which X is SO₂ and Y is CH₂ are obtainable by oxidation of thioethers (4): 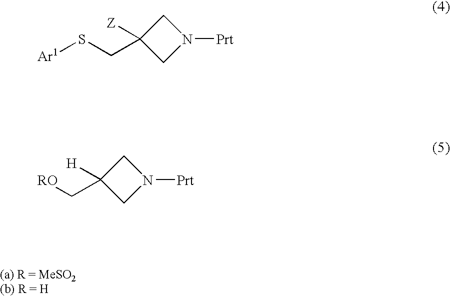
where Z and Ar¹ have the same meanings as before. Suitable oxidants include Oxone™, in methanol at 0° C. to ambient temperature.

Thioethers (4) in which Z is H are obtainable by treatment of mesylates (5a) with thiophenols Ar¹SH (eg in refluxing acetonitrile in the presence of potassium carbonate), where Ar¹ has the same meaning as before. Mesylates (5a) are obtained by treatment of hydroxymethyl derivatives (5b) with methanesulfonyl chloride in an inert solvent such as dichloromethane in the presence of base such as triethylamine.

Preferred 5HT2A receptor antagonists are:
1-[2-(2,4-difluorophenyl)ethyl]-3-{[(4-fluorophenyl)sulfonyl]methyl}azetidine;
1-(4-fluorophenyl)-2-(3-{[(4-fluorophenyl)sulfonyl]methyl}azetidin-1-yl)ethanone;
1-(4-fluorophenyl)-4-(3-{[(4-fluorophenyl)sulfonyl]methyl}azetidin-1-yl)butan-1-one hydrochloride;
2-(3-{[(2,4-difluorophenyl)sulfonyl]methyl}azetidin-1-yl)-1-(4-fluorophenyl)ethanone hydrochloride;
2-(3-fluoro-3-{[(4-fluorophenyl)sulfonyl]methyl}azetidin-1-yl)-1-(4-fluorophenyl)ethanone hydrochloride;
1-[2-(2,4-difluorophenyl)ethyl]-3-{fluoro[(4-fluorophenyl)sulfonyl]methyl}azetidine hydrochloride;
3-{difluoro[(4-fluorophenyl)sulfonyl]methyl}-1-[2-(2,4-difluorophenyl)ethyl]azetidine hydrochloride;
6-fluoro-3-[(3-{[(4-fluorophenyl)sulfonyl]methyl}azetidin-1-yl)methyl]-1,2-benzisothiazole hydrochloride;
6-fluoro-3-[(3-{[(4-fluorophenyl)sulfonyl]methyl}azetidin-1-yl)methyl]-1,2-benzisoxazole hydrochloride; and
1-(4-fluoro-2-methylphenyl)-2-(3-{[(4-fluorophenyl)sulfonyl]methyl}azetidin-1-yl)ethanone;
and pharmaceutically acceptable salts thereof.

The 5HT2A receptor antagonists may be used alone or in combination with each other.

Suitable D2 receptor antagonists for use according to the invention include, for example, amisulpride, aripiprazole, benperidol, chlorpromazine, clozapine, flupentixol, fluphenazine, haloperidol, levopromazine, olanzapine, pericyazine, perphenazine, pimozide, prochlorperazine, promazine, quietapine, remoxipride, risperidone, sertindole, sulpiride, trifluoroperazine, thioridazine, thiothixene, ziprasidone, zotepine and zupenthixolclozapine, and pharmaceutically acceptable salts thereof.

Preferred D2 receptor antagonists are chlorpromazine, haloperidol, trifluoroperazine, thiothixene and fluphenazine, and pharmaceutically acceptable salts thereof.

The D2 receptor antagonists may be used alone or in combination with each other.

The skilled person will appreciate that a compound displaying dual 5HT2A and D2 receptor antagonism, such as risperidone, ziprasidone, iloperidone, melperone and olanzapine, will also be suitable for use in the present invention where a 5HT2A receptor antagonist and a D2 receptor antagonist are required.

Furthermore, the skilled person will appreciate that a compound displaying triple NK3, 5HT2A and D2 receptor antagonism will also be suitable for use in the present invention where a NK3 receptor antagonist, a 5HT2A receptor antagonist and a D2 receptor antagonist are required. An example of such a compound is N-{(S)-2-(3,4-dichlorophenyl)-4-[3-(2-fluorophenyl)-8-aza-bicyclo[3.2.1]oct-8-yl]-butyl}-3,5,N-trimethyl-benzamide.

It will be appreciated by those skilled in the art that certain of the compounds described herein may exist in stereoisomeric forms (i.e. they may contain one or more asymmetric carbon atoms or may exhibit cis-trans isomerism). The individual stereoisomers (enantiomers and diastereomers) and mixtures of these are included within the scope of the present invention.

Compounds for use according to the invention may be administered simultaneously or sequentially and, when administration is sequential, the NK3 receptor antagonist, 5HT2A receptor antagonist and D2 receptor antagonist may be administered in any order.

Compounds for use according to the invention may be administered as the raw material but the active ingredients are preferably provided in the form of pharmaceutical formulations.

The active ingredients may be used either as separate formulations or as a single combined formulation. When combined in the same formulation it will be appreciated that the active ingredients must be stable and compatible with each other and the other components of the formulation. Therefore, pharmaceutical formulations comprising a combination as defined above together with a pharmaceutically acceptable diluent or carrier comprise a further aspect of the invention. When formulated separately they may be provided in any convenient formulation, conveniently in such manner as are known for such compounds in the art.

In a further aspect, the present invention provides the use of a NK3 receptor antagonist, or a pharmaceutically acceptable derivative thereof, in combination with a 5HT2A receptor antagonist, or a pharmaceutically acceptable derivative thereof, and/or a D2 receptor antagonist, or a pharmaceutically acceptable derivative thereof, for the manufacture of a medicament for use in the treatment of schizophrenia and associated conditions.

Conditions associated with schizophrenia include, not only the symptoms of schizophrenia, but also, for example, schizophreniform disorders, schizoaffective disorders, psychotic disorders and delusional disorders. Other associated conditions include, for example, disorders involving psychoses, including Alzheimer's disease, psychotic major depression, Parkinson's disease and bipolar disorder.

It will be appreciated that reference to treatment is intended to include prophylaxis as well as the alleviation of established symptoms.

In a further aspect, the invention provides a method of treatment of a mammal, including a human, suffering from schizophrenia or conditions associated with schizophrenia comprising administering a combination of a NK3 receptor agonist, or a pharmaceutically acceptable derivative thereof, with a 5HT2A receptor antagonist, or a pharmaceutically acceptable derivative thereof, and/or a D2 receptor antagonist, or a pharmaceutically acceptable derivative thereof.

In one embodiment of the invention, there is provided a method of treatment, as hereinbefore described, wherein the mammal is a human who has not responded to treatment using a NK3 receptor antagonist, or a 5HT2A receptor antagonist, or a D2 receptor antagonist alone.

In a further embodiment of the invention, there is provided a method of treatment, as herein before described, wherein the mammal is a human who has not responded to treatment using 5HT2A receptor antagonist in combination with a D2 receptor antagonist, either as two separate actives or as one dual activity drug.

In a further aspect, the invention provides a pharmaceutical composition for use in the treatment of schizophrenia and/or conditions associated with schizophrenia comprising a NK3 receptor antagonist, or a pharmaceutically acceptable derivative thereof, with a 5HT2A receptor antagonist, or a pharmaceutically acceptable derivative thereof, and/or a D2 receptor antagonist, or a pharmaceutically acceptable derivative thereof.

In a further aspect, the invention provides a pharmaceutical composition for use in the treatment of schizophrenia and/or conditions associated with schizophrenia comprising a NK3 receptor antagonist, or a pharmaceutically acceptable derivative thereof, with a 5HT2A receptor antagonist, or a pharmaceutically acceptable derivative thereof, and/or a D2 receptor antagonist, or a pharmaceutically acceptable derivative thereof, characterized in that the effective dose of NK3 receptor antagonist is lower than when the NK3 receptor antagonist is administered alone.

The NK3 receptor antagonist, 5HT2A receptor antagonist and/or D2 receptor antagonist may be co-administered in the form of separate pharmaceutical compositions for simultaneous and/or sequential use. Preferably, the NK3 receptor antagonist, 5HT2A receptor antagonist and/or D2 receptor antagonist are administered as a single pharmaceutical composition comprising effective amounts of the active ingredients.

A pharmaceutically acceptable formulation of the invention can allow for the effective distribution of the compositions of the instant invention in the physical location most suitable for their desired activity. Non-limiting examples of agents suitable for formulation with the compositions of the instant invention include: biodegradable polymers, such as poly (DL-lactide-coglycolide) microspheres for sustained release delivery after intracerebral implantation (Emerich, DF et al, 1999, Cell Transplant, 8, 47-58 (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The present invention also includes pharmaceutically acceptable compositions prepared for storage or administration, which include the desired compounds in a pharmaceutically acceptable carrier, diluent, or adjuvant. Acceptable carriers or diluents for therapeutic use are well known in the pharmaceutical art, and are described, for example, in Remington's Pharmaceutical Sciences, Mack Publishing Co. (A. R. Gennaro edit. 1985), incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically acceptable composition or formulation is in a form suitable for administration into a subject. Suitable forms, in part, depend upon the use or the route of entry, for example oral, transdermal, or by injection. Such forms should not prevent the composition or formulation from reaching a target cell or organ. For example, pharmacological compositions injected into the blood stream should be soluble. Other factors are known in the art, and include considerations such as toxicity and forms that prevent the composition or formulation from exerting its effect.

A pharmaceutically acceptable formulation of the invention can be delivered to a subject by a liposome delivery mechanism. Standard protocols for formation of liposomes can be followed. The compositions of the present invention can also be formulated and used as for example, tablets, capsules or elixirs for oral administration, suppositories for rectal administration, sterile solutions, suspensions for injectable or inhalation administration.

The pharmaceutically acceptable formulations can be locally delivered by, for example, direct injection or by use of an infusion pump. Direct injection, such as subcutaneous, intramuscular, or intradermal injection, can take place using standard needle and syringe methodologies, or by needle-free technologies such as those described in Conry et al., 1999, Clin. Cancer Res., 5, 2330-2337 and Barry et al., International PCT Publication No. WO 99/31262.

Compositions of the invention can be delivered to a subject by systemic administration. Systemic administration is in vivo systemic absorption or accumulation of drugs in the blood stream followed by distribution throughout the entire body. Administration routes that can lead to systemic absorption include, without limitation: intravenous, subcutaneous, intraperitoneal, inhalation, transdermal, oral, intrapulmonary and intramuscular.

The compositions of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intradermal, intramuscular, or intrathecal injection or infusion techniques and the like.

The pharmaceutical compositions can be in a form suitable for oral use, for example, as tablets, troches, lozenges, aqueous or oily suspensions, dispersible powders or granules, emulsions, hard or soft capsules, or syrups or elixirs. Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient or ingredients in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in a mixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutically acceptable compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The compositions of the invention can also be administered in the form of suppositories, e.g., for rectal administration of the drugs. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compositions can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The amount of NK3 receptor antagonist used according to the present invention will depend on several factors such as the seriousness of the disease, the individual response of the patient, the kind of formulation or the route of administration, but it is usually comprised between 0.01 mg and 10 mg per kg of body weight divided into a single dose or into more daily doses, e.g. 2, 3 or 4 times daily. For a 70 kg adult, the total daily dose will normally be in the range of 1 to 500 mg, more preferred 1 to 200 mg. In the case of a unit dose (which may be administered singly or if desired in multiples to achieve the desired total daily dose), the unit dose may contain from 0.25 to 500 mg, in particular 0.5, 1, 2.5, 5, 7.5, 10, 15, 20, 50, 100, 150, or 200, 250, 300, 350, 400, 450 or 500 mg, more preferred being those dosages less than or equal to 200 mg.

The amount of 5HT2A receptor antagonist used according to the present invention is about 0.01 to 250 mg/kg per day, preferably about 0.05 to 100 mg/kg per day, and especially about 0.05 to 5 mg/kg per day. The compounds may be administered on a regimen of 1 to 4 times per day.

The amount of D2 receptor antagonist used according to the present invention is about 0.1 to 10,000 mg/day, preferably about 0.25 to 1,000 mg/day, more preferably about 0.5 to 500 mg/day, especially about 1 to 200 mg/day. In the case of a unit dose (which may be administered singly or if desired in multiples to achieve the desired total daily dose), the unit dose may contain from 0.1 to 500 mg, in particular 0.25, 0.5, 1, 2.5, 5, 10, 25, 50, 100, 150, 200, 250, 300, 400 or 500 mg, more preferred being those dosages less than or equal to 200 mg.

1-11. (canceled)

12. A method for the treatment of a patient suffering from schizophrenia or a condition associated with schizophrenia which comprises administering to the patient an effective amount of a NK3 receptor antagonist, or a pharmaceutically acceptable derivative thereof, and a 5HT2A receptor antagonist, or a pharmaceutically acceptable derivative thereof.

13. A method for the treatment of a patient suffering from schizophrenia or a condition associated with schizophrenia which comprises administering to the patient an effective amount of a NK3 receptor antagonist, or a pharmaceutically acceptable derivative thereof, and a D2 receptor antagonist, or a pharmaceutically acceptable derivative thereof.

14. A method for the treatment of a patient suffering from schizophrenia or a condition associated with schizophrenia which comprises administering to the patient an effective amount of a NK3 receptor antagonist, or a pharmaceutically acceptable derivative thereof, and a 5HT2A receptor antagonist, or a pharmaceutically acceptable derivative thereof, and a D2 receptor antagonist, or a pharmaceutically acceptable derivative thereof.

15. The method of claim 12 wherein the NK3 receptor antagonist is selected from talnetant, osanetant, or a pharmaceutically acceptable salt thereof.

16. The method of claim 12 wherein the 5HT2A receptor antagonist is selected from olanzapine, risperidone, ziprasidone, pizotifen, iloperidone, ritanserin, ketanserin, cyproheptadine, aripiprazole, arylipiperazines, risperidone metabs, ACP-103, AT-1015, Org-5222, AR-116081, MDL-100907, RP 71602, IT-657, F-94116-CN, GMC-283, EMR-62218, or a pharmaceutically acceptable salt thereof.

17. The method of claim 12 wherein the compounds of the combination are administered simultaneously or sequentially.

18. The method of claim 12 wherein the conditions associated with schizophrenia are selected from the symptoms of schizophrenia, schizophreniform disorders, schizoaffective disorders, psychotic disorders, delusional disorders, disorders involving psychoses, Alzheimer's disease, psychotic major depression, Parkinson's disease and bipolar disorder.

19. The method of claim 13 wherein the NK3 receptor antagonist is selected from talnetant, osanetant, or a pharmaceutically acceptable salt thereof.

20. The method of claim 13 wherein the D2 receptor antagonist is selected from amisulpride, aripiprazole, benperidol, chlorpromazine, clozapine, flupentixol, fluphenazine, haloperidol, levopromazine, olanzapine, pericyazine, perphenazine, pimozide, prochlorperazine, promazine, quietapine, remoxipride, risperidone, sertindole, sulpiride, trifluoroperazine, thioridazine, thiothixene, ziprasidone, zotepine, zupenthixolclozapine, or a pharmaceutically acceptable salt thereof.

21. The method of claim 13 wherein the compounds of the combination are administered simultaneously or sequentially.

22. The method of claim 13 wherein the conditions associated with schizophrenia are selected from the symptoms of schizophrenia, schizophreniform disorders, schizoaffective disorders, psychotic disorders, delusional disorders, disorders involving psychoses, Alzheimer's disease, psychotic major depression, Parkinson's disease and bipolar disorder.

23. The method of claim 14 wherein the NK3 receptor antagonist is selected from talnetant, osanetant, or a pharmaceutically acceptable salt thereof.

24. The method of claim 14 wherein the 5HT2A receptor antagonist is selected from olanzapine, risperidone, ziprasidone, pizotifen, iloperidone, ritanserin, ketanserin, cyproheptadine, aripiprazole, arylipiperazines, risperidone metabs, ACP-103, AT-1015, Org-5222, AR-116081, MDL-100907, RP 71602, IT-657, F-94116-CN, GMC-283, EMR-62218, or a pharmaceutically acceptable salt thereof.

25. The method of claim 14 wherein the D2 receptor antagonist is selected from amisulpride, aripiprazole, benperidol, chlorpromazine, clozapine, flupentixol, fluphenazine, haloperidol, levopromazine, olanzapine, pericyazine, perphenazine, pimozide, prochlorperazine, promazine, quietapine, remoxipride, risperidone, sertindole, sulpiride, trifluoroperazine, thioridazine, thiothixene, ziprasidone, zotepine, zupenthixolclozapine, or a pharmaceutically acceptable salt thereof.

26. The method of claim 14 wherein the compounds of the combination are administered simultaneously or sequentially.

27. The method of claim 14 wherein the conditions associated with schizophrenia are selected from the symptoms of schizophrenia, schizophreniform disorders, schizoaffective disorders, psychotic disorders, delusional disorders, disorders involving psychoses, Alzheimer's disease, psychotic major depression, Parkinson's disease and bipolar disorder.
